(19) United States
(12) Patent Application Publication
(10) Pub. No.: US 2007/0260480 A1
(43) Pub. Date: Nov. 8, 2007

(54) Managing health related variables in a remote data collection system

(76) Inventor: Johan Cederlund, Bromma (SE)

Correspondence Address: HARNESS, DICKEY & PIERCE, P.L.C., P.O. BOX 8910, RESTON, VA, 20195, US

(21) Appl. No.: 11/417,204

(22) Filed: May 4, 2006

(51) Int. Cl. G06Q 10/00; A61B 5/00

(52) U.S. Cl. 705/2; 600/300

(57) ABSTRACT

A method and system of managing health related variables in a system for collecting data from one or more remote units to a central unit is disclosed. A set of health related variables is defined for which data should be collected from a remote unit associated with an individual and the set of health related variables is sent from the central unit to the remote unit.

### FIELD OF THE INVENTION

The invention relates to a system for collection of data with respect to health related variables from remote units to a central unit and a method and apparatus for use in such a system.

### BACKGROUND OF THE INVENTION

There are a number of factors influencing the effect of a medical treatment of a patient, such as treatment by means of a medication. For example the body constitution, other medications, food and drink, physical activity might influence the effects of a medication for a specific patient. The effect of the medication will vary largely between patients and over time for some conditions and hence, for these conditions, individual medication schemes need to be designed and adjusted over time.

In order to achieve good effects of a medication, the dosage of the medication and/or interval between intake of the medication need to be adjusted. Sometimes also the type of medication is adapted. Traditionally the adjustments have been made in response to monitoring the effects of the medication for a specific patient by means of regular follow up visits to a physician at which the patient is asked a number of questions e.g. regarding how the effect of the medication has varied over time since the last follow up visit.

The traditional monitoring requires frequent follow up visits in order for the patient to be able to give details about the situation since the last follow up visit. To be able to decrease the frequency of follow up visits and to enhance the precision in the monitoring, systems have been suggested for collecting data with respect to the patient between the follow up visits. For example systems have been suggested where data indicating effect of the medication and other variables such as physical activity etc are being collected between follow up visits e.g. in a personal digital assistant (PDA) and that this collected data may be collected by the physician at the follow up visits. An example of such a system is disclosed in U.S. Pat. No. 5,672,154.

Also systems have been suggested where medical data is collected from a remote unit of the patient by means of wireless transmission of the medical data to a central unit. Such a system is disclosed in US patent application No. 2005/0187789.

Still there is a need for further enhancement of the monitoring of each individual patient.

### SUMMARY OF THE INVENTION

This disclosure relates to a system for collection of data from remote units to a central unit and specifically to managing of health related variables in such a system.

A set of health related variables are defined for which data should be collected from a remote unit associated with an individual, such as a patient, and the set of health related variables are sent from the central unit to the remote unit.

By sending the set of health related variables from the central unit to the remote unit, the set of variables to use may be provided to the remote unit without a need to predefine them in the remote unit and/or without the need for the remote unit and/or the individual to be at the location of the central unit when receiving the set of health related variables.

The set of health related variables may for example be sent from the central unit to the remote unit via a wireless connection.

It is to be noted that even though the set of health related variables are said to be sent from the central unit to the remote unit, the transfer may be initiated either by the central unit or by the remote unit, i.e. using a push or pull function.

Also, updating of the health related variables in the remote unit is enabled. An updated set of variables can be defined and it can be sent to the remote unit. The updating of the health related variables may e.g. be done in response to receiving data with respect to the current set of health related variables sent from the remote unit. This functionality may be provided together with the functionality of sending all variables of a set of health related variables or separately.

### BRIEF DESCRIPTION OF THE DRAWINGS

In the following embodiments of the invention will be described in relation to the environment and systems in which they may be advantageously employed, and with reference to the enclosed drawings on which:

FIG. 1 shows a block diagram of a system in which embodiments of the invention may be employed,

FIGS. 2a and 2b shows display images used for data input in an embodiment of the invention,

FIG. 3 shows a flow chart of a method of an embodiment of the invention,

FIG. 4 shows a flow chart of a method of another embodiment of the invention, and

FIG. 5 shows a block diagram of a remote unit according to an embodiment of the invention.

### DETAILED DESCRIPTION OF THE PREFERRED EMBODIMENTS

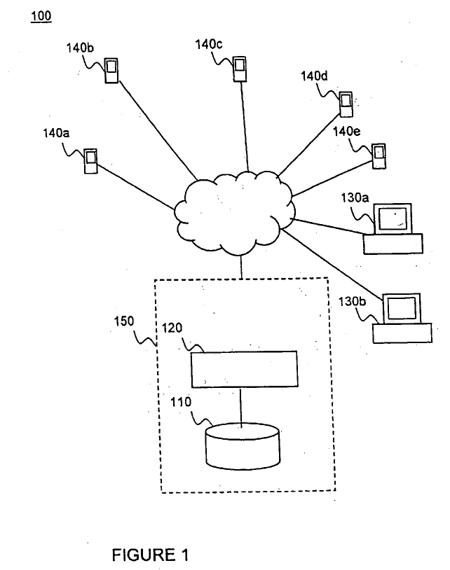

FIG. 1 shows a block diagram of a system 100 in which embodiments of the invention may be advantageously employed. The system 100 is an interactive system for continuous follow up of the health-situation of patients, e.g. in order to monitor the effect of one or more types of medications which the patients are taking.

The system 100 may be used to identify an individual medication scheme for one or more patients. In such a case, the system uses continuous registrations of data made by the patients regarding their health-situation. The scheme for registration is normally based on their particular diagnosis and choices made by a physician. The patients use remote units, e.g. in the form of mobile phones, for registration, and the physician and/or nurse use PC's and Internet to view and evaluate the patients' health-status development.

The system 100 may also be used to perform medical studies on a set of subjects taking a medication. In such a case the system uses continuous registrations of data made by the patients regarding their health-situation and taking of medication.

The system may also be used in several other applications for monitoring the health-situation of a person over time, such as monitoring a person's weight reduction e.g. in view of prescribed physical activity and/or diet.

Schematically, the system 100 in FIG. 1 consists of three parts:

a database 110 in combination with a server 120 where all information and patient registrations are stored,

an Internet application with which the physicians and nurses (and patients if they are allowed to) are communicating, using Internet Explorer or a similar tool on their PC 130a-b, and

a mobile application installed on a patient's mobile phone 140a-e and used by the patient to make registrations, and communicating with the Internet application.

The database 110 and Internet application are located in a central unit 150. To this end, the term “central” is not primarily referring to a physical location, but to the fact that the central unit 150 is a logical center of the system 100 and collects data from all of the remote units 140a-e, whereas the remote units 140a-e, with respect to this application, generally only communicates with the central unit 150. The central unit 150 in itself may be located in one physical unit but may just as well be distributed in separate physical units. An instance of the mobile application is located in each remote unit 140a-e. Although the remote units 140a-e are disclosed as mobile phones in FIG. 1, they may be any form of communication device which enables sending of data to the central unit 150 from a remote location, such as a stationary or portable computer with internet access.

The communication between the mobile application, e.g. installed on the patient's mobile phone 140a-e, and the Internet application, may be completely automatic and accomplished using the cellular mobile system for transportation of data. The information may be sent as soon as the registration is finished, enabling for the healthcare personnel to see it short after the registration has been concluded. However, the invention does not exclude the possibility for the data to be stored and sent at a later occasion. This may for example be suitable in a case where the remote unit is a wireless unit and there is no coverage for wireless transmission at the time when the data is input.

The central unit can be put together in several different ways but a typical way is to have a web server that fronts the database. The web server accepts remove calls over https or http (depending on security preferences) at a specific path.

The remote unit is preferably a unit that can communicate over https or http, e.g. mobile phones, lap-tops, PDA, etc.

In the case of mobile phones, the phone can transfer the data in several different ways. Either the mobile phone transfers the data formatted in a specific XML-format over https or http via the cellular network (GPRS), or the communication is based on SMS. Another example could be a WAP-interface.

PDA:s could communicate with the central unit either via https or http with a built-in wireless internet access (several standards exists, e.g. IEEE 802.11a/b/g etc.), or by synchronizing the PDA using the PDA:s cradle and then use the internet access from the PC to access the central unit.

Lap-tops communicate with the central unit via the internet access in the lap-top. It could either be a wireless (several standards exists, e.g. IEEE 802.11a/b/g etc.) or wired access.

Data are collected with respect to a number of health related variables. The term “health related variable” refers to a factor which is relevant to a person's health and which may vary over time. Such health related variables are of interest e.g. when monitoring the effect of a medication, or other prescriptions such as physical activity or diet, on a patient (or subject). A health related variable can e.g. be a patient experienced/subjective condition or a measurable/objective condition, or an activity. Examples of patient experienced conditions are pain, fatigue, mood, etc. Examples of measurable conditions are blood pressure, blood sugar level, ECG , EEG, weight etc. Examples of activities are diet, sleep, physical strain, taking of medication etc.

Depending on the health related variable, the data input may be of different formats. For example, for a patient experienced condition, such as pain, the input may be a subjectively relative value, such as a scale from no pain to unbearable pain. For a condition which can be measured, such as blood pressure, the input may be a numerical value.

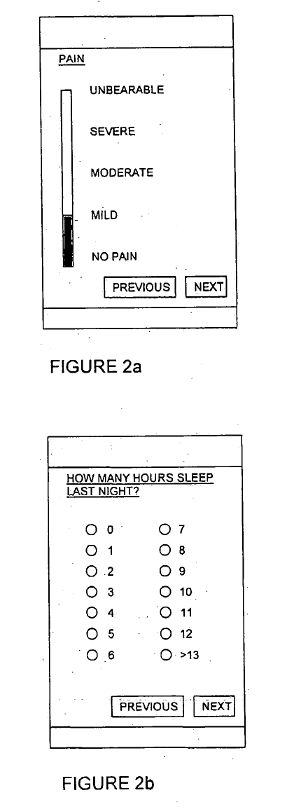

Data for a specific health related variable is normally collected by stating the health related variable or by stating a question with respect to the health related variable. The patient then provides data with respect to the health related variable, e.g. by selecting one of a number of options given, or by providing a numerical or textual input. For example, as shown in FIG. 2a, the health related variable “Pain” is shown on the display of the mobile phone of the patient together with a scale indicating “unbearable”, “severe”, “moderate”, “mild” and “no pain”. In this case the patient would respond by indicating the pain on that scale. In another example shown in FIG. 2b, the question “How many hours did you sleep last night?” referring to the health related variable ‘sleep’ is being shown on the display. In this case the patient would respond by input of a numeric value, e.g. by using the key pad of the mobile phone or by selecting one of a number of given alternatives given on the display.

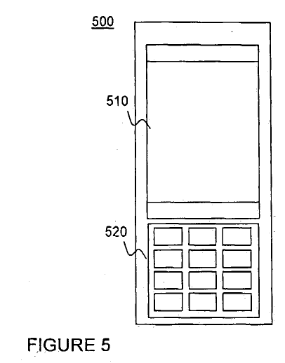

A schematic view of a mobile phone 500 used as a remote unit is shown in FIG. 5. The presentation of the questions with respect to or the name of the variable and options for responding is shown on a display 510 and the input from the patient can be made by means of a key pad 520 of the mobile phone.

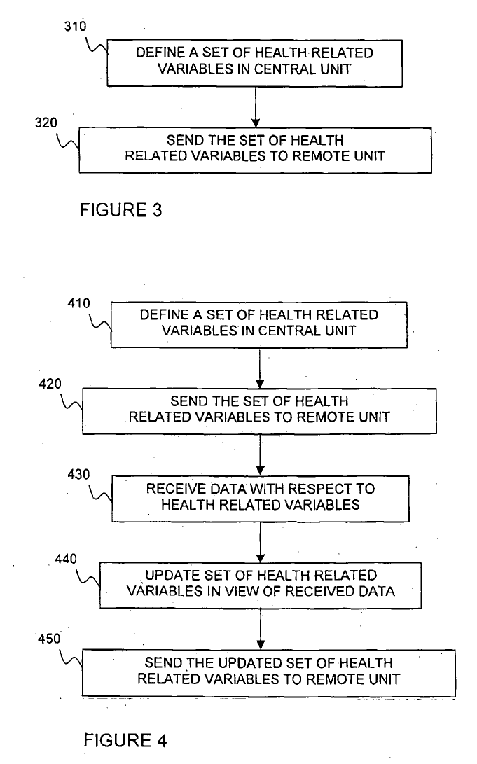

In FIG. 3, a flow chart of a method according to an embodiment of the invention is shown. The method may be implemented in the system disclosed in FIG. 1. Data for a set of health related variables should be collected from a remote unit associated with a patient.

In the case where the method is used for designing an individualized medication scheme, the set of health related variables is normally selected by a physician based on the diagnosis of the patient, general rules and first observations of the patient.

In the case where the method is used in a study of a medication and its effects, the set of health related variables is normally selected by the person or organization performing the study and includes health related variables of interest in the study.

The set of health related variables is defined in the central unit in a step 310, e.g. by creation of new variable posts, and/or by selection of predefined variables in the system in which the method is implemented.

After the set of health related variables has been defined, the set of health related variables is sent from the central unit to the remote unit in a step 320.

In the case a variable is already known to the remote unit, e.g. since it has been predefined or already transferred to the remote unit, the variable need only be sent in the form of an identifier identifying the known variable. If the variable is not known to the remote unit, a more full description of the variable need to be sent.

Once the set of health related variables have been transferred to the remote unit, a similar function may be used to manage updating of variables. An updated set of health related variables are defined in the central unit. The updating may include adding of health related variables, omission of health related variables or both to the set of health related variables.

After the defining in the central unit, the updated set of health related variables is sent from the central unit to the remote unit.

As for the case for transmitting the set of health related variables, the sending of the updated set of variables may be realized in different ways. For example, if the updating involves an omission of a variable, the updated set of health variables need only be sent in the form of an identifier identifying the omitted variable. In the case a health related variable is added and the variable is already known to the remote unit, e.g. since it has been predefined or already transferred to the remote unit, the updated set of health variables may similarly only be an identifier identifying the added variable. In the case an added variable is not known to the remote unit, the updated set of variables needs to be sent in the form of a more full description of the added variable.

In FIG. 4 another embodiment of the invention is shown. The first two steps 410 and 420 correspond to the steps 310 and 320 of FIG. 3. The central unit then receives data with respect to the set of health related variables sent from the remote unit as defined in the central unit in a step 430. In view of this data the set of variables defined in the central unit may be updated in a step 440 and the updated set of variables is then sent from the central unit to the remote unit in a step 450 as described above.

Also, in response to the received data, a medication scheme for the patient associated with the remote unit may be updated and the updated medication scheme can be sent from the central unit to the remote unit. The medication scheme defines the type of medications, the amount of each medication and the time schedule for the taking of each medication.

In addition to a patient having a remote unit in which data for the set of health related variables is input and sent to the central unit, a further remote unit may be associated to the patient. For example, someone living together with the patient or other who can observe the patient may be given a remote unit.

In such a case, a further set of health related variables is defined for which data should be collected from the further remote unit associated with the patient. The further set of health related variables is then sent from the central unit to the further remote unit.

It is to be noted that this feature does not actually require two different remote units. The same remote unit may be used as long as it is possible to separate the data input by the patient and the data input by the other person. Similarly, the further set of health related variables may include different variables than the set of health related variables but may just as well include the same variables.

In addition to defining the set of health related variables, a number of other features of the collection of data can be defined remotely.

For example, one such feature of the data collection process is a time schedule for prompting input of data with respect to the set of health related variables into the remote unit. The time schedule can be defined in the central unit and sent to the remote unit in a similar fashion as the set of health related variables as described above. For example that data collection could be prompted at certain times during the day, e.g. 8.00 am, 1.00 pm and 6.00 pm. Data may be collected with respect to different health related variables at the different times during the day is also envisaged.

Another example of such a feature of the data collection process is the format of data to be collected with respect to a health related variable. In the central unit the format of data is defined for each variable of the set of health related variables. The format of data is then sent from the central unit to the remote unit. As for the sending of the set of health related variables, a complete description of the format may be sent to the remote unit. In an alternative embodiment, a categorizing of health related variables in variable categories is used. Each variable category defines the format of data to be collected with respect to the health related variable. By predefining the variable categories and their implications in the remote unit, the variable category of the health related variable need only be sent from the central unit to the remote unit in order to define in the remote unit the format of data to be collected from the remote unit to the central unit. The variable category may also give basic information of the format of a display image to be used as an interface to the user of the remote unit for collecting data with respect to the health related variable belonging to the a variable category.

The set of health related variables for which data should be collected, the format of the data to be collected for each variable, and the time schedule for the input of the data for each variable together with the format of a display image used as an interface to the user of the remote unit constitute a form for data collection with respect to the health related variables. According to embodiments of the invention, parts of or all of the form can be transferred to the remote unit from the central unit and parts of or all of the features of the form in the remote unit may be updated remotely from the central unit.

The variables are defined in the central unit 150 by means of variable defining means, such as by means of an operator, such as a physician, using a keyboard or other interface means to define the set of health related variables. The set of health related variables, is sent to a remote unit from the central unit 150 by means of sender, such as a modem or other interface means via the internet and e.g. a mobile network in case of the remote unit being a mobile communication device, such as a mobile phone. Similarly, the central unit 150 receives data with respect to the set of health related variables from the remote unit by means of a suitable receiver, preferably by means of the same communication networks as the set of health related variables were sent.

It is to be noted that even if embodiments have been described where the set of health-related variables and the time schedule is defined and updated in the central unit, the invention does not exclude the possibility for the user of the remote unit to update the set of health related variables, time schedule for prompting or other parts of the form. For example, the time schedule may not be suitable for a patient, e.g. by prompting the patient before the patient has woken up in the morning. In such a case the patient may in certain cases delay the prompting with a certain amount of time. Also, the alternatives for input of data may not reflect the patients situation. In such a case the user may in certain cases add alternatives.

Another advantage which may be achieved by using a system as disclosed in FIG. 1, is that the compliance to a prescribed medical treatment by means of a medicine may be enhanced. More specifically, a patient will be reminded by the application in the remote unit to take the medication. The patient will further be prompted to input data with respect to a set of health related variables. The patient may then be given feedback indicating the results of the medicine.

For example, the data to be input by the patient may include information of whether the patient has taken the medicine or not, i.e. information regarding compliance. The feedback may then indicate a difference in the data regarding other health related variables than compliance in cases where the patient complies with the prescription and cases where the patient does not.

Also, prompting of input of data and the real time properties of the input of data with respect to health related variables increases a patient's active participation in the process of developing an individual medication scheme and may also increase the awareness of the effects of the different health related variables mutual relationship.

The use of a system of the type shown in FIG. 1 also enables more frequent follow up of a patient's health situation, more frequent adaptation the health variables monitored, and more frequent adaptation of the patient's individual medication scheme, e.g. by a physician or other medical staff. This will in turn enable an increased activity and awareness of the medical staff of the effects of the different health related variables, the mutual relationship between the different health related variables, and the effects of different medication schemes etc.

Furthermore, the system of FIG. 1 will enable automatic decisions based on the data collected in the central unit from a remote unit. For example, the data collected may result in an automatic decision to change the set of health-related variables for which data are to be collected and also to change the medication scheme or even the type of medicine for the patient.

In one implementation of the system of FIG. 1, every health related variable has a unique identifier. The health related variables are represented in the data model by a number of attributes:

Id

Name

Output vector

Data Type

Server Editor

Client Editor

Max Concentration

Minimal Increment

Extension In Time

Unit

The Output Vector is the type of the variable (variable category).

The Data Type collected for the variable can take the following values: integer, integer string, float, string.

The Server Editor is the recommended editor type for the variable on the server side (central unit).

The Client Editor is the recommended editor type for the variable on the client side (remote unit).

The Max Concentration is the maximum value for the variable.

Minimal Increment is the increments in which the value of the variable can be changed.

Unit is the unit that measures the value of the variable.

Every variable has a number of translations. A variable that either has a number of discrete values or whose range of values is described by a stepped scale has a number of children describing the discrete values or steps.

The schedule defines groups of variables that are made available for registration to the patients. Every schedule has a unique identifier.

The first group is the variables that are available for spontaneous registration of variables, every variable is also designated as mandatory or voluntary for the spontaneous registration.

The rest of the groups define sets of variables that will be registered at times during the day. Depending of the Output Vector type of the variable, recommendations can be made for the variable.

Every patient is identified by an identifier. Every patient is allocated a number of variables for which data will be collected. A unique schedule is setup for the patient to collect the data for the variables. More than one schedule can exist for a patient but only one schedule can be active for any given day.

The client periodically initiates an update operation of variable and schedule definitions. The client contacts the server identifying itself using the patient identifier and the current schedule identifier. The server checks the database to see if a new schedule exists for the patient. If one exists the server compiles the set of variable definitions used in the schedule and the groups of variables defined in the schedule together with the user defined translations for the variables and returns them to the client. The client updates the local database with the variable and schedule definitions.

The way a variable is presented to a patient is decided by the following:
1. The Mobile Editor attribute of the Variable
2. The Output Vector attribute of the Variable
3. The Unit attribute of the Variable

The Mobile Editor attribute controls what type of user interface control to be shown to the patient.

The Output Vector attribute controls whether a recommended value will be shown to the patient.

The Unit attribute controls the measurement unit shown to the patient.

In an alternative to a system where data are collected to the central unit with respect to a set of health related variables, the data may be used locally at the remote unit. For example, a user of a remote unit may initiate a download of a set of health related variables and optionally a time schedule for prompting input of data with respect to the variables. The data input may then be stored and used locally at the user, either in the remote unit or another unit of the user serving as a central unit. Such a method may e.g. be used for dieting scheme, where a user of a remote unit downloads a dieting scheme to a remote unit which will then prompt the user to input data with respect to a set or health related variables, such as food eaten, weight, etc. at given times. The remote unit or the central unit of the user may then include an application for processing the input data, give feedback to the user, alter the dieting scheme etc.

1. A method of managing health related variables in a system for collecting data from one or more remote units to a central unit, comprising:
defining a set of health related variables for which data should be collected from a remote unit associated with an individual; and
sending the set of health related variables from the central unit to the remote unit.

2. The method of claim 1, wherein the set of health related variables is sent from the central unit to the remote unit via a wireless connection.

3. The method of claim 1, further comprising:
defining an updated set of health related variables; and
sending the updated set of health related variable from the central unit to the remote unit.

4. The method of claim 1, further comprising:
receiving data with respect to the set of health related variables sent from the remote unit;
updating the set of variables in response to the received data; and
sending the updated set of health related variable from the central unit to the remote unit.

5. The method of claim 1, further comprising:
defining a further set of health related variables for which data should be collected from a further remote unit associated with the individual; and
sending the further set of health related variables from the central unit to the further remote unit.

6. The method of claim 1, further comprising:
receiving data sent with respect to the set of health related variables from the remote unit;
in response to the received data, updating a medication scheme for the individual associated with the remote unit; and
sending the updated medication scheme from the central unit to the remote unit.

7. The method of claim 1, further comprising:
defining a time schedule for prompting input of data with respect to the set of health related variables into the remote unit, and
sending the time schedule from the central unit to the remote unit.

8. The method of claim 7, further comprising:
defining an updated time schedule for prompting input of data with respect to the set of health related variables into the remote unit, and
sending the updated time schedule from the central unit to the remote unit.

9. The method of claim 1, further comprising:
defining the format of data to be collected with respect to a health related variable, and
sending the format of data from the central unit to the remote unit.

10. The method of claim 1, further comprising:
categorizing a health related variable in a variable category, said variable category defining the format of data to be collected with respect to the health related variable, and
sending the variable category of the health related variable from the central unit to the remote unit.

11. The method of claim 1, further comprising:
defining, in the remote unit, an updated set of health related variables.

12. The method of claim 7, further comprising:
defining, in the remote unit, an updated time schedule for prompting input of data with respect to the set of health related variables into the remote unit.

13. An apparatus for managing health related variables in a system for collecting data from one or more remote units, comprising:
a variable defining means for defining a set of health related variables for which data should be collected from a remote unit associated with an individual; and
a sender for sending the set of health related variables to the remote unit.

14. The apparatus of claim 13, wherein the sending means is further arranged for sending the set of health related variables to the remote unit via a wireless connection.

15. The apparatus of claim 13, wherein the variable defining means is further arranged for defining an updated set of health related variables and the sender is further arranged to send the updated set of health related variable to the remote unit.

16. The apparatus of claim 13, further comprising:
a receiver for receiving the data with respect to the set of variables sent from the remote unit, and wherein
the defining means are further arranged define an updated set of health related variables in response to the received data and the analysis thereof, and
the sender is further arranged to send the updated set of health related variables to the remote unit.

17. The apparatus of claim 13, wherein the defining means is further arranged for defining a further set of health related variables for which data should be collected from a further remote unit associated with the individual, and said sender is further arranged for sending the further set of health related variables to the further remote unit.

18. The apparatus of claim 13, wherein the defining means comprises a user interface for defining the set of health related variables.

19. The apparatus of claim 13, wherein the defining means is further arranged to define a time schedule for prompting input of data with respect to the set of health related variables into the remote unit, and the sender is further arranged to send the time schedule to the remote unit.

20. The apparatus of claim 13, wherein the defining means is further arranged to define an updated time schedule for prompting input of data with respect to the set of variables into the remote unit, and the sender is further arranged to send the updated time schedule to the remote unit.

21. The apparatus of claim 13, wherein the defining means is further arranged to define the format of data to be collected with respect to a health related variable, the sender is further arranged to send the format of data from the central unit to the remote unit.

22. The apparatus of claim 13, wherein the defining means are further arranged to categorize a health related variable in a variable category, said variable category defining the format of data to be collected with respect to the health related variable, and the sender is arranged to send the variable category of the health related variable from the central unit to the remote unit.

23. An apparatus arranged for sending data to a central unit, comprising:
a receiver for receiving, from the central unit, a set of health related variables for which data should be sent;
a user interface for inputting data with respect to the set of health related variables; and
a sender for sending input data to the central unit.

24. The apparatus of claim 23, wherein the receiver is further arranged for receiving, from the central unit, an updated set of health related variables and the user interface is further arranged for inputting data with respect to the updated set of variables.

25. The apparatus of claim 23, wherein the receiver is further arranged for receiving, from the central unit, a time schedule for prompting input of data with respect to the set of variables, and the user interface is further arranged for prompting input of data according to the time schedule.

26. The apparatus of claim 23, wherein the receiver is further arranged to receive the format of data to be sent with respect to a health related variable, said user interface is arranged to prompt input of data with respect to the health related variable on the format of data, and the sender is arranged to send input data with respect to the health related variable on the format of data to the central unit.

27. The apparatus of claim 23, wherein the receiver is arranged to receive a variable category with respect to a health related variable, said variable category defining the format of data to be collected with respect to the health related variable, said user interface is arranged to prompt input of data with respect to the health related variable on the format of data, and the sender is arranged to send input data with respect to the health related variable on the format of data to the central unit.

28. The apparatus of claim 23, wherein the receiver is a receiver for wireless communication.

29. The apparatus of claim 23, wherein said user interface is further arranged for inputting an updated set of health related variables.

30. The apparatus of claim 29, wherein said user interface is further arranged for inputting an updated time schedule for prompting input of data with respect to the set of health related variables into the remote unit.

31. A system for collecting data with respect to health related variables from a remote unit associated with an individual to a central unit, wherein:
the remote unite comprises:
a user interface for inputting data with respect to a set of health related variables; and
a sender for sending input data to said central unit,
and the central unit comprises:
a receiver for receiving the data,
wherein the central unit further comprises:
a defining means for defining a set of health related variables for which data should be collected; and
a sender for sending the set of health related variables to the remote unit,
and wherein the remote unit further comprises:
a receiver for receiving the set of health related variables.
